(19) United States
(12) Patent Application Publication
(10) Pub. No.: US 2014/0186824 A1
(43) Pub. Date: Jul. 3, 2014

(54) DYNAMIC BIOCHEMICAL TISSUE ANALYSIS ASSAYS AND COMPOSITIONS

(71) Applicants: Monica Maki Burdick, Athens, OH (US); Doug Goetz, Athens, OH (US); Ventesh Sridmar Shirure, Athens, OH (US); Ramiro Malgor, Athens, OH (US); Vicente Resto, Galveston, TX (US)

(72) Inventors: Monica Maki Burdick, Athens, OH (US); Doug Goetz, Athens, OH (US); Ventesh Sridmar Shirure, Athens, OH (US); Ramiro Malgor, Athens, OH (US); Vicente Resto, Galveston, TX (US)

(73) Assignee: Ohio University, Athens, OH (US)

(21) Appl. No.: 14/130,083

(22) PCT Filed: Jun. 29, 2012
(86) PCT No.: PCT/US2012/044836

§ 371 Date: Feb. 17, 2014

Related U.S. Application Data
(60) Provisional application No. 61/503,973, filed on Jul. 1, 2011.

(51) Int. Cl. G01N 33/569

(52) U.S. Cl. CPC G01N 33/56966
USPC 435/6.1; 435/29; 435/7.21; 530/387.3

(57) ABSTRACT

Described herein is a method, and compositions useful with the method, of analyzing a tissue from a subject. The method includes contacting a probe conjugate (10) including a probe molecule (12), to a biological tissue 146 from a subject. The probe conjugate (10) is contacted with the biological tissue (14) under a well defined and easily controlled force field(s) and that tends to result in an interaction between the molecular probe (12) and the tissue (14) and/or tends to disrupt such interactions. The resulting interaction of the molecular probe (12) with the tissue (14) is then quantified. The analytical methods may be useful for diagnostic and prognostic applications as well as in the discovery of novel drug delivery systems and novel therapeutic substances and targets.

### FIELD OF THE INVENTION

The present invention is directed to assays and compositions for analyzing tissues and more particularly to a dynamic biochemical tissue analysis assay.

### BACKGROUND OF THE INVENTION

Histology is the study of tissue derived from plants, animals, insects, or humans and is ubiquitous in bioengineering, life science research, and the development of novel biotechnologies. One of the more powerful techniques used in histology is the biochemical analysis of the tissue. In biochemical tissue analysis, the biochemistry and cell biology of the tissue is characterized by exploring the binding of a molecular probe (e.g. peptide, antibody, poly nucleotide) to a tissue section. The results of such an assay are often used to diagnose, predict the outcome of, and/or develop a therapeutic path for, a disease that is present within the organism from which the section was derived.

In addition, histology is germane to the development of bioengineered tissues, the manufacture of bioproducts from plant and animal tissue, understanding host response to artificial surfaces, the diagnosis and selection of therapy for animal, human, insect, or plant disease, and studies aimed at gaining fundamental insight into the molecular mechanisms of physiological processes. Central to histology is the probing of tissue with antibodies and peptides, an endeavor often referred to as immunohistochemistry (IHC). Typically the goal of IHC is to determine if a certain antigen to the probe molecule is present on or within the tissue.

While the number of IHC protocols is enormous and highly variable, they all follow the same general outline. In a typical assay, the tissue section is subjected to a solution containing a molecular probe that is cognate to the antigen of interest. Subsequently, the original solution is removed and the tissue exposed to a series of developing solutions that reveal the presence of the molecular probe, bound to the tissue via molecular bonds, as intensely colored reaction products. Detection of the molecular probe indicates the presence of the antigen on the tissue. The endpoint of the assay is typically highly qualitative with results often expressed as ++ (for very positive), + (for somewhat positive) and − (for no detection of probe). A conceptually similar assay is in situ hybridization where the probing molecule is a nucleic acid. For the purposes of this invention disclosure, we define “biochemical tissue analysis” to be any assay that uses a molecular probe to characterize tissue from plants, animals, insects, and humans including but not limited to IHC that use antibodies as the probe and in situ hybridization type assays that use nucleic acids as the probe.

The results of biochemical tissue analysis are dictated by the biophysics of the probe-antigen bond and the conditions under which the probe and antigen are brought and maintained in contact. A key factor governing the results of biochemical tissue analysis is the biophysics of the probe-antigen bond. For assays done with soluble probes in the fluid phase, the most relevant biophysical parameter for traditional protocols is the bond affinity. The results of the analysis are dictated by the interplay between the bond properties and the external conditions imposed on the probe-antigen bond. For example, after the probe is incubated with the tissue, the tissue is typically washed multiple times to remove “unbound” probe. The amount of probe removed with each wash is directly related to the affinity of the probe for the antigen as well as the affinity of the probe for non-target components of the tissue. Typically one or both of these affinities are not known. That said, the experimentalist will frequently choose the probe with the highest affinity for the target antigen since this choice presumably increases the likelihood of observing differences between the probes binding to the target antigen relative to binding to non-target regions of the tissue. Thus, most experimentalists are interested in identifying only high affinity interactions associated with traditional biochemical tissue analysis. The techniques employed with standard biochemical tissue analysis are not useful for identifying, observing, or quantifying bonds under the broad range of conditions affecting target-probe interactions, specifically applied forces. Therefore, techniques and compositions are needed to allow exploration of bond interactions under dynamic, well-controlled force conditions.

Much progress has been made in automating and standardizing biochemical tissue analysis (e.g. Leica Microsystem's extensive product line of automated tissue sectioning/preparation instruments and attempts to use image analysis to quantify the endpoint). That said, the full power of biochemical tissue analysis is unrealized due to the fact that (a) only a single type of interaction, a high affinity interaction between the probe and the tissue section, is explored and (b) it is difficult to control and systematically vary the force placed on the probe-antigen bond. It is abundantly clear from decades of work in cell adhesion that biomolecular interactions are governed by a plethora of factors that can only be discovered and appreciated by tightly controlling and varying the contact between the participating molecules. Considering biochemical tissue analysis in light of this knowledge, reveals that traditional biochemical tissue analysis can be considered a one-dimensional assay which leaves vast regions of the multi-dimensional interaction space unexplored. In addition, the currently used assays are typically not, in general, standardized—the interaction of the probing molecule and the surface is not tightly controlled, nor is it easily controlled, in traditional biochemical tissue analysis.

Crude assays exist which suggest the power and need for force-controlled biochemical tissue analysis. The use of tissue sections to probe for fluid shear-dependent interactions was established over 30 years ago in the Stamper-Woodruff assay, before the identification of the selectins as a unique class of shear-sensitive adhesion molecules. Mouse lymphocytes expressing what we now know as L-selectin were overlaid on glass-immobilized mouse lymph node frozen tissue sections. These slides were placed on an orbital shaker and subjected to non-quantifiable rotational shear. After a brief incubation period, slides were quickly yet carefully removed from the shaker and fixed in gluteraldehyde for observation by light microscopy. The mouse lymphocytes were found to adhere specifically to lymph node high endothelial venules only when subjected to rotational shear and not under static (no flow) conditions, thereby revealing a unique set of shear-dependent lymph node homing molecules. More recently, it has been reported that the Stamper-Woodruff technique could also be used to investigate cancer cell line adhesion to E-selectin on vascular endothelial cells of frozen mouse liver sections. Such studies highlight the potential usefulness of more advanced histology assays, in particular those involving shear. However, the Stamper-Woodruff assay is extremely subject to human error, in that any disruption in shear, particularly as slides are removed from the orbital shaker to the fixative solution, results in complete failure of the assay as bound cells detach from the tissue surface. Furthermore, the inability to deliver controlled shear stress results in a qualitative assay, unable to reflect a decade's worth of reports that discrete fluid shear levels exquisitely regulate adhesion molecule function. Clearly, such tissue-based assays like the Stamper-Woodruff have merit, but experimental conditions must be stringently controlled in order to collect data of value. The dynamic biochemical tissue analysis assay described in this disclosure addresses significant shortcomings in the Stamper-Woodruff assay as well as traditional biochemical tissue analysis.

### SUMMARY OF THE INVENTION

Described herein is a method, and compositions useful with the method, of analyzing a tissue from a subject. The method includes contacting a tissue with a probe conjugate that includes a probe molecule conjugated to an inert surface. A force is applied to the probe conjugate, the tissue, or both the probe conjugate and the tissue. The force is sufficient to result in the interaction of the probe conjugate with the tissue. The resulting interaction of the probe conjugate with the biological tissue is then characterized and quantified. The inert surface may be in the form of a particle, a generally planar surface, a projection, and combinations thereof. The inert surface may include one or more probe molecules. The method may be used in diagnostic and prognostic methods.

### BRIEF DESCRIPTION OF THE DRAWINGS

The accompanying drawings, which are incorporated in and constitute a part of this specification, illustrate various embodiments of the invention and, together with a general description of the invention given above and the detailed description of the embodiments given below, serve to explain the embodiments of the invention.

FIG. 1A is an illustration of an embodiment of a probe conjugate in accordance with embodiments of the invention.

FIG. 1B is an illustration of an embodiment of a probe conjugate in accordance with embodiments of the invention.

FIG. 1C is an illustration of an embodiment of a probe conjugate in accordance with embodiments of the invention.

FIG. 2 is a graph illustrating data obtained in accordance with embodiments of the invention.

FIG. 3 is a photomicrograph of a probe conjugate (in this case spherical particles) interacting with a tissue in accordance with embodiments of the invention.

FIG. 4 is a graph illustrating data obtained in accordance with embodiments of the invention.

FIG. 5A is a photomicrograph of a probe conjugate (in this case spherical particles) interacting with a tissue in accordance with embodiments of the invention.

FIG. 5B is a photomicrograph of a probe conjugate (in this case spherical particles) interacting with a tissue in accordance with embodiments of the invention.

FIG. 5C is a photomicrograph of a probe conjugate (in this case spherical particles) interacting with a tissue in accordance with embodiments of the invention.

FIG. 5D is a photomicrograph of a probe conjugate (in this case spherical particles) interacting with a tissue in accordance with embodiments of the invention.

FIG. 6A is a graph illustrating the rolling velocity of a spherical probe conjugate in accordance with embodiments of the invention.

FIG. 6B is a graph illustrating the rolling velocity of a spherical probe conjugate in accordance with embodiments of the invention.

FIG. 7A is a contour graph of the data from FIG. 6A in accordance with embodiments of the invention.

FIG. 7B is a contour graph of the data from FIG. 6B in accordance with embodiments of the invention.

FIG. 8A is a photomicrograph of an immunohistochemical analysis of the tissues sections used to obtain the data in FIG. 6A.

FIG. 8B is a photomicrograph of an immunohistochemical analysis of the tissues sections used to obtain the data in FIG. 6A.

FIG. 8C is a photomicrograph of an immunohistochemical analysis of the tissues sections used to obtain the data in FIG. 6A.

FIG. 8D is a photomicrograph of an immunohistochemical analysis of the tissues sections used to obtain the data in FIG. 6A.

FIG. 9 is a graph illustrating data obtained in accordance with embodiments of the invention.

### DETAILED DESCRIPTION OF THE INVENTION

The dynamic biochemical tissue analysis assay, in accordance with embodiments of the invention, greatly expands traditional biochemical tissue analysis into an assay which systematically investigates a multitude of interactions between a probe molecule and a tissue section under well controlled conditions. In addition to allowing tight control over the conditions of the analysis, dynamic biochemical tissue analysis (DBTA) allows identification of force-sensitive interactions between the probe molecule and the tissue sections. Previously used standard biochemical analysis methods are not capable of systematically characterizing force-sensitive aspects of target-probe interactions. Force-sensitive aspects of biochemical bonds have been implicated in a host of disease processes including pathological inflammation, heart disease, and cancer.

In DBTA, a molecular probe is conjugated to an inert surface (e.g. a particle of any shape, generally planar support or an end portion of a projection extending from a base structure). Exemplary molecular probes may be any molecular entity that can serve as an ligand (or antigen) for a molecule that may be expressed on a tissue section and may include an amino acid, a peptide, a polypeptide, an antibody, fragments of antibodies, a protein, glycoproteins, a proteoglycan, a carbohydrate, a polysaccharide, a nucleotide, a polynucleotide, an oligonucleotide, a RNA, a DNA, a peptide nucleic acid, a lipid, a glycoplipid, a siRNA, a miRNA, a small molecule, and combinations thereof.

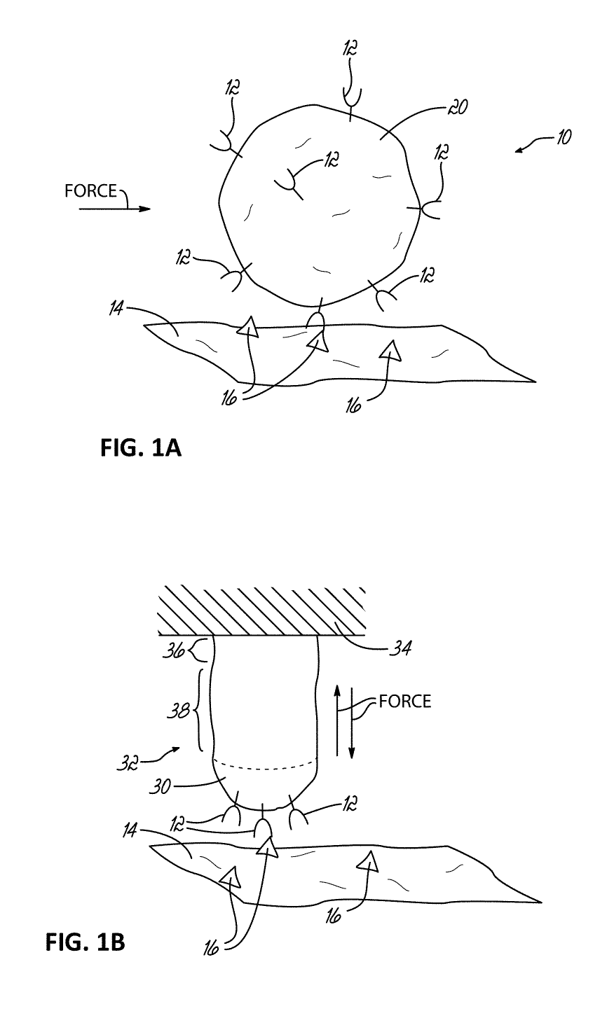

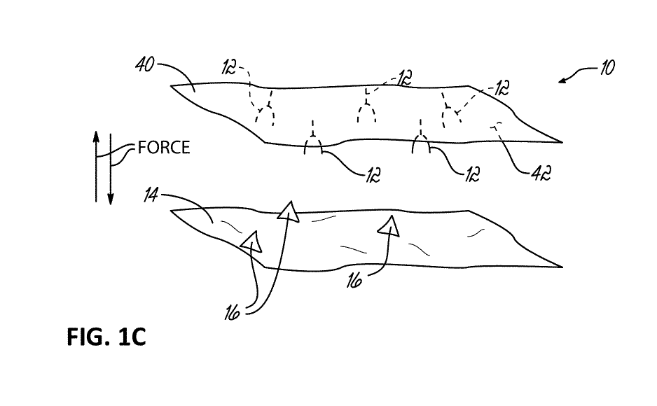

With reference to FIGS. 1A-1C, the inert surface 10 may have any form that allows the molecular probes 12 to interact with the tissue 14 as well as the characterization and evaluation of such interaction. Exemplary forms include individual particles 20, a tip portion 30 of a projection 32 extending from a base structure 34, or a generally planar support 40.

The individual particles 20 can have any shape that allows for the interaction of the molecular probe 12 with the tissue 14, such as an antigen 16 on the tissue 14, as well as the characterization and evaluation of such interaction. In an exemplary embodiment, the particles 20 are generally spherical. The particles have a size sufficient to allow the molecular probe to interact with a tissue while under the influence of relatively small but well controlled and well defined forces. In an embodiment, the particles may have a largest diameter in a range from about 1 nanometer to about 100 micrometers. In another embodiment, the particles have a largest diameter in a range between about 100 nanometers to about 50 micrometers. In another embodiment, the particles have a largest diameter in a range between about 1 micrometer and about 40 micrometers. The particle 20 could have one species of molecular probe or multiple species of molecular probes.

The projection 32 has a base portion 36 coupled to a base structure 34, a tip portion 30 conjugated with molecular probes 12, and an intermediate portion 38 extending between the base portion 36 and the tip portion 30. The base portion 36, the tip portion 30, and the intermediate portion 38 may each be made of the same material or different materials. The tip portion 30 may be rounded, pointed, flattened, or enlarged relative to the intermediate portion. The projection 32 has a size sufficient to allow the molecular probe 12 to interact with a tissue 14, such as an antigen 16 on the tissue 14, while under the influence of relatively small but well controlled and well defined forces. The projection 32 has a length extending along the longitudinal axis between the base portion 36 and the tip portion 30 in the range between about 500 nanometers and about 2 micrometers and a largest width perpendicular to the longitudinal axis that is in the range between about 1 micrometer and about 40 micrometers. In another embodiment, the length is in the range between about 1 micrometer and 10 micrometers and the width is in the range between about 20 micrometers and about 100 micrometers. The tip portion 30 could have one species of molecular probe or multiple species of molecular probes. In one embodiment, tip portion 30 could range in size from 1 nanometer to 20 nanometers in diameter, and in another embodiment, tip portion 30 could range in size from 1 micron to 100 micrometers in diameter.

The generally planar support 40 has a generally flat contact surface 42 to which the molecular probes 12 are conjugated such as by micropatterning. The generally planar support 40 could have one species of molecular probe or multiple species of molecular probes arranged in a known pattern.

The inert surface may be made from a variety of materials including polystyrene, ceramics, proteins, polymers such as biodegradable polymers, synthetic polymers, and biological polymers, magnetic material(s), electrically active material(s), liposomes, polymersomes, micelles, proteoglycans, quantum dots, metal containing polymers, metals, ultrasound bubbles—essentially any material including those that have salient optical properties, e.g., fluorescent materials and/or any combinations of the above mentioned materials. The molecular probe may be conjugated to the inert surface using routine techniques known to those skilled in the art. In an embodiment, the molecular probe is conjugated to the inert surface via a linker molecule. In another embodiment, the molecular probe is directly conjugated to the inert surface. Exemplary techniques for conjugating a molecular probe to an inert surface are non-specific adsorption of the probe onto the inert surface, or specific recognition of ligand (immunoglobulin Fc of a molecular probe) to a receptor (protein A) coated on an inert surface. The molecular probe is conjugated to the inert surface at a density sufficient to allow for the characterization of the interaction between a desired probe and a tissue. The density of the molecular probe may vary depending on the probe and the target antigen, and the form of the inert surface being evaluated. Those skilled in the art will be able to determine the optimal density of the molecular probe to be conjugated to the inert surface.

In one embodiment of the method, the single type of inert surface is conjugated to a single type of molecular probe. In another embodiment of the method, the single type of inert surface is conjugated to multiple types of molecular probes. In another embodiment of the method, multiple inert surfaces of the same type or different types are conjugated to one or more types of molecular probes. In another embodiment of the method, multiple sets of inert surfaces of the same type that can be identified as distinct (such as through the use of different colors of particles such as green polystyrene particles, red polystyrene particles, blue polystyrene particles, each with the same shape and size) are each conjugated with a distinct molecular probe.

In use, the probe conjugates are brought into contact with a tissue section under a well characterized force. The force may tend to result in the interaction of the molecular probe with the tissue and also may tend to result in the disruption of the interaction of the molecular probe with the tissue. Thus, in certain cases, the well characterized force may be thought of as two forces, an associative force and a disruptive force. Some molecular probes, such as selectins, have enhanced interactions with a tissue under the influence of a force. Other molecular probes may not require a force to interact with a tissue, but the associative force may still bring the molecular probe into contact with the tissue to result in the interaction. The disruptive force tends to result in the disruption of the interaction between the molecular probe and the tissue. That is, the disruptive force tends to disrupt the bonds that form between the molecular probe and the ligand on the tissue. While the disruptive force tends to disrupt the interaction of the molecular probe with the tissue, the disruption does not necessarily have to result in the disruption of the interaction. For example, in some instances the disruptive force may not be sufficient to disrupt the interaction between the molecular probe and the tissue. In such an instance, these data would still be valuable in characterizing the interaction between the molecular probe and the tissue. In some embodiments, the associative force and the disrupting force may be the same well characterized force that in the first instance facilitates the interaction and in the second instance results in the disruption of the interaction between the probe conjugate and the tissue. In another embodiment, the associative force and the disruptive force are distinct forces. The associative force and the disruptive force may be applied in the same or different directions and/or may be the same or different intensity. In one embodiment, the force is in the range from about 0.1 dynes per square centimeter to about 200 dynes per square centimeter. In another embodiment, the force is in the range between about 1 dyne per square centimeter and about 10 dynes per square centimeter. In an alternative embodiment, the force is in a range between about 1 piconewton to about 1 millinewton. In another alternative embodiment, the force is in a range between about 100 piconewtons to about 100 micronewtons.

The force may be in the form of a force field, a mechanical force, a gravitational force, a centrifugation force, a magnetic field, optical force, electrical field, acoustic force or combinations thereof. In an embodiment, a force field can be realized by a fluid flow field such as by mounting the tissue section in a parallel plate flow chamber, using spherical particles as the inert surface, such polystyrene particles, perfusing the particles through the flow chamber, and thus realizing a well characterized fluid shear force on the particles as they interact with the tissue section. In other embodiments, a force field can be realized with a magnetic field, an electric field, by an optical force (such as with the use of lasers that can apply a force and move particles) or an acoustic force employed with particles that are susceptible to such forces. For example, iron containing particles would be susceptible to a magnetic force.

In an embodiment, a single particle type is used in an assay. In another embodiment, multiple types of particles may be used simultaneously in a single assay. Similarly, each type of particle may have one or multiple species of molecular probe(s). The probe(s) on the different types of particles may be the same or different from the ones on the other types of particles used in the assay.

High through-put assays can be achieved by utilizing tissue section arrays, multiple particles (each bearing one or more unique molecular probes), in conjunction with microfluidic and nanofabricated chambers that have multiple channels thus affording the multi-probe analysis of tens to hundreds, to even thousands of tissue sections in a single assay.

In another embodiment, the inert surface is a nano to micron sized tip portion of a projection extending from a base structure. The tip portion may have any shape and can be conjugated with probe molecule(s) and brought into contact with the tissue section under well-controlled force conditions that move the tip portion towards or away from the tissue section such as by a mechanical force. A single projection could be used or multiple projections could be mounted on a surface to create a probe “stamp”. Each tip portion for the projections could have one or more species of molecular probe. Each tip in a stamp may have the same molecular probe profile as the other tips or a molecular probe profile that differs from the other tip portions in the stamp.

In another embodiment, the inert surface is a micropatterened surface that can be conjugated with probe molecule(s) via micropatterning and the surface brought into contact with the tissue section under well-controlled force conditions that move the micropatterned surface towards or away from the tissue section. The micropatterned surface could have one species of molecular probe or tens, hundreds or even thousands of distinct molecular probes. In an embodiment, quantification can include determining the force needed to displace the micropatterned surface from the tissue section once it has made contact with the tissue section. In another embodiment, quantification includes the force needed to drive the micropatterned surface into the tissue section. In another embodiment, quantification includes observing perturbations in an electrical, magnetic, or force field due to interactions between the tissue and the micropatterned surface.

The device generating the force may generate one or more force (such as one or more mechanical force or force field) and these force(s) can be modified (e.g. increased, decreased or set to zero) throughout the course of the assay. One could also utilize the naturally occurring gravitational force field in the assay (e.g. using particles that are more dense than the suspending buffer thus resulting in a net gravitational force on the particles) independently or in conjunction with a device that generates a force field.

The interaction of the probe conjugates with a tissue can be observed in real time such as by placing the device on a suitable microscope stage (e.g. an inverted phase contrast microscope). In the alternative, images of the tissue section and the probe conjugate can also be recorded (such as via a video recorder and stored in a digital medium such as on a DVD or a computer hard drive) and the generated recorded images evaluated at a later time. The evaluation may be conducted either manually or automatically. Manual evaluation includes observation by a researcher who quantifies and characterizes the interaction of the probe conjugate with the tissue section. Automatic evaluation includes processes, such as computer based processes that analyze the images to generate data quantifying and characterizing and the interaction of the probe conjugate with the tissue section. Combinations of automatic and manual quantification and characterization may also be employed.

In the case of using particles, quantification includes identifying the number of particles that are adherent to the tissue section and their adhesive nature which may be determined by characterizing the interaction of the particles with the tissue section, such as identifying particles as firmly adherent, rolling, skipping etc. and/or determining their translational or rotational velocities. Additional exemplary methods of quantifying the adherence of particles to the tissue section include measuring the amount of time that a particle adheres to a tissue section, or measuring the velocity with which particles move over the surface of a tissue sample, or the number of times a particle interacts with a tissue section. In all cases, the quantification can be done on a single particle, a subset of adherent particles or all of the adherent particles. In all cases the quantification can be performed as a function of the force exerted on the particles and the position of the particles. The analysis can be done with the aid of mathematical models that describe the particle transport in the chamber, the biophysics of the adhesive interactions of the particles with the tissue sections or adhesive substrates, and/or models that describe the force field(s) present within the assay. If optically active particles are used (e.g. fluorescent particles) then the level of luminescence/absorbance from the tissue, post-exposure to the particle probes, can be quantified. Likewise if electrically active particles are employed then the conductivity (or resistivity) of the tissue, post-exposure to the particle probes, can be quantified. If magnetically active particles are used then the response of the tissue, post-exposure to the particle probes, to a magnetic field can be quantified.

In the embodiments using a projection tip, a probe stamp, or a micropatterned surface, quantification can include: determining the force needed to displace the tip(s) or micropatterned surface from the tissue section once it has made contact with the tissue section; the force needed to drive the tip(s) or micropatterned surfaces into the tissue section; detection of the deflection of the tip(s); perturbation in an electrical, magnetic or force field due to interaction(s) between the tissue and the tip(s) or micropatterned surfaces. In the case of a stamp, these measurements can be made for each individual tip within the stamp. The data generated from the experimental conditions can be compared with appropriate controls to provide insight into the biochemistry of the tissue section.

Embodiments of the invention may also be used in diagnostic or prognostic assays, or to design novel drug delivery systems or treatments. For example, the results of an assay may be correlated with the ultimate outcome of the subject from which the sample was derived, (e.g. did the subject have a metastatic event). Correlating the data from a number of subjects could be used to establish criteria that could be used for diagnostic and prognostic assays for other subjects, to find molecules that could be targeted by novel drug delivery mechanisms, and/or to identify molecules to be targeted by therapeutic treatments in other subjects. The term “subject” is understood to include any source of biological tissue including, without limitation, mammals (e.g., human and non-human mammals), non-mammalian animals (e.g. reptiles, amphibians, and fish), insects, and/or plants.

This technique, i.e. Dynamic Biochemical Tissue Analysis (DBTA), has numerous salient features including the following: (i) it allows investigation of force-sensitive aspects of interactions between the probe and the tissue (current assays cannot capture these force-sensitive behaviors), (ii) it allows the experimentalist to easily characterize and vary the way in which the probe contacts the tissue (current approaches are typically not standardized and quite difficult to standardize) and (iii) it generates a well-defined force on the probe-antigen bond thus allowing detailed characterization of the probe-antigen bond (e.g. determining the bond's reactive compliance).

### EXAMPLES

As demonstrated in this example, embodiments of the inventive dynamic biochemical tissue analysis process reveal force-sensitive interactions between the molecular probe and the tissue section.

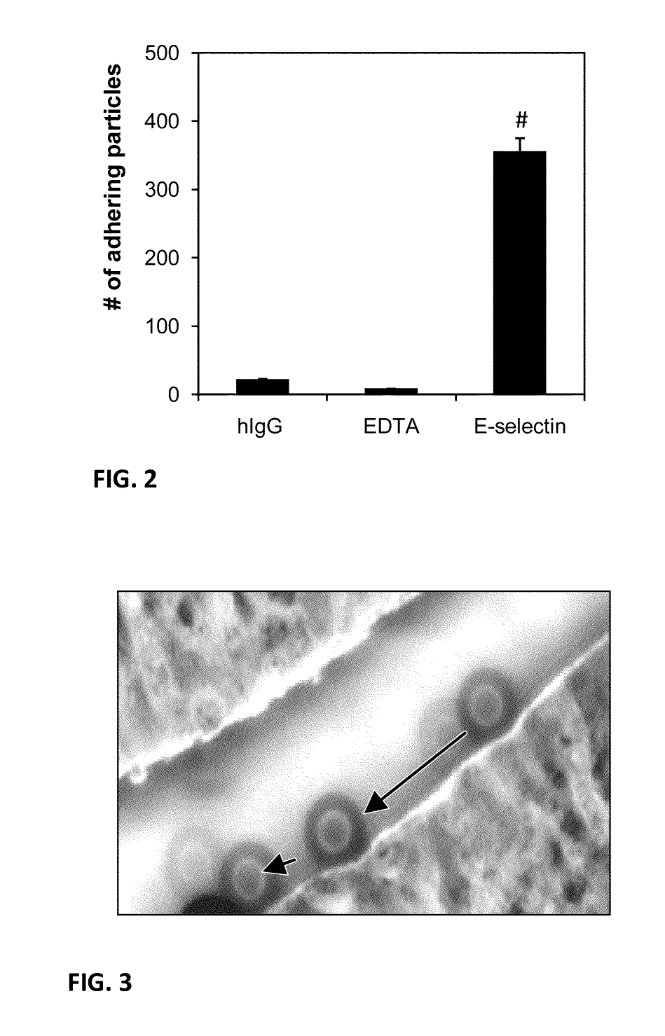

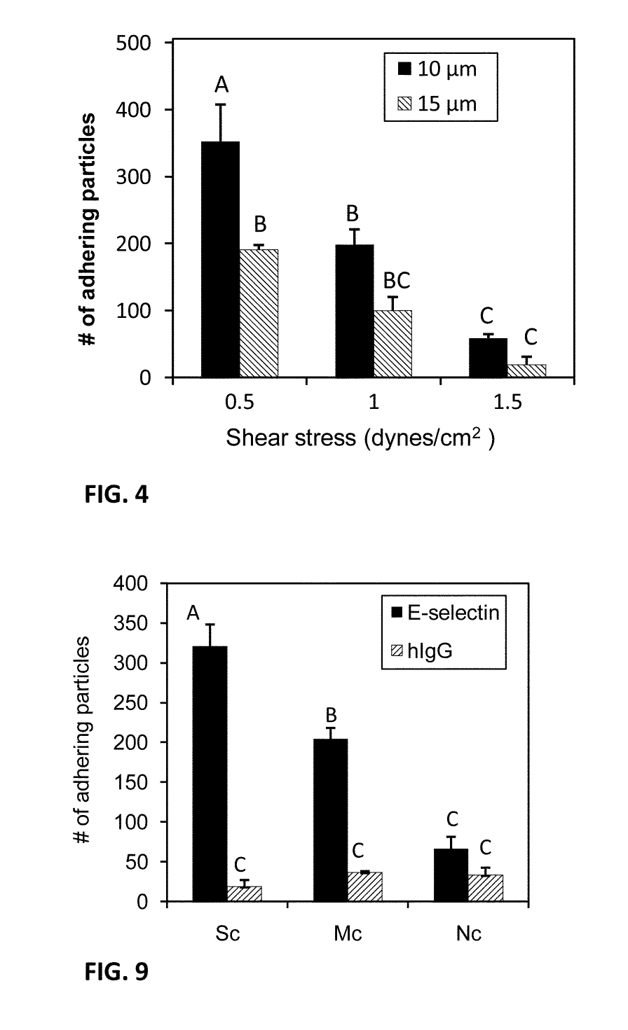

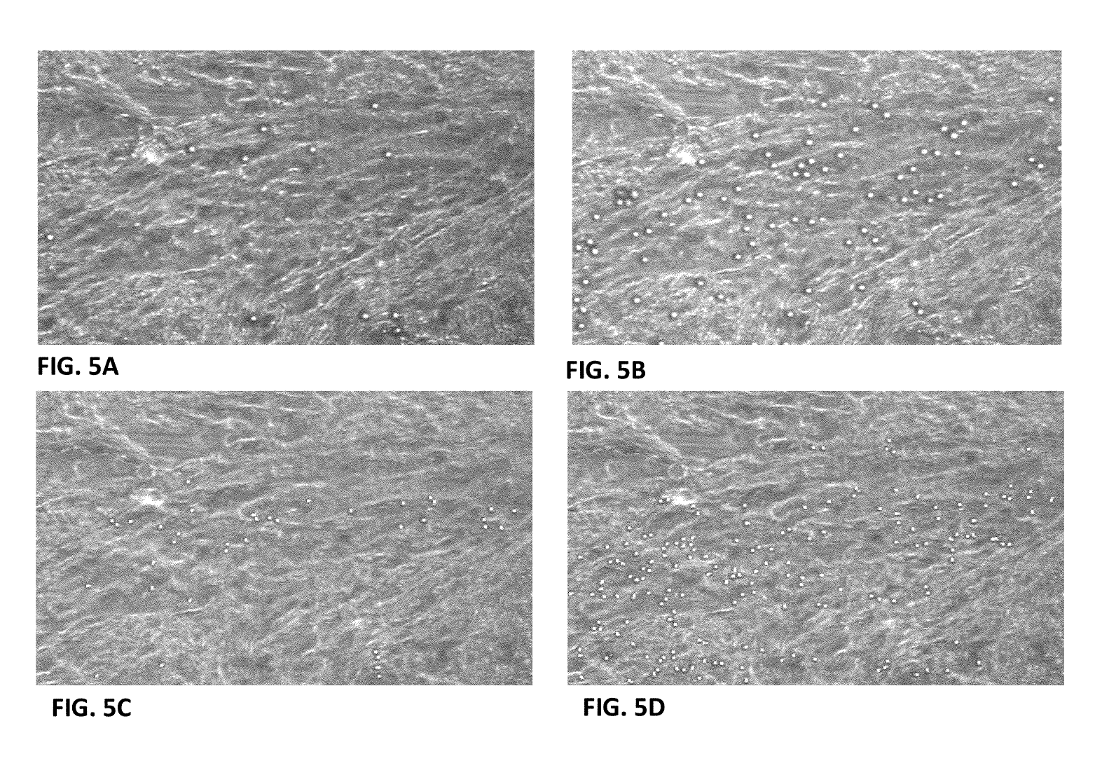

For this example, fifteen micron diameter microspheres were conjugated with an E-selectin-IgG chimera. As one negative control, microspheres were conjugated with human IgG (hIgG). For this study, E-selectin or hIgG microspheres [15 μm polystyrene particles prepareded with 10 μg/ml recombinant mouse E-Selectin-IgG chimera (E-selectin) or human IgG (hIgG) for 2 hrs and blocked with 1% BSA] in buffer containing Ca²⁺ and Mg²⁺, or E-selectin microspheres suspended in 10 mM EDTA (EDTA bar in figure) were perfused over tissue derived from invasive adenocarcinoma of the colon at 1 dynes/cm² in the DBTA assay. The flow chamber was mounted on an inverted phase contrast microscope connected to a video camera which in turn was linked to a computer that captured the images. It was observed that only the E-selectin-IgG microspheres exhibited a significant interaction with the tissue sections (FIG. 2, Data are mean±SEM for n=3; # P<0.0001 (Tukey's multiple comparison test) relative to either hIgG microspheres or E-selectin microspheres in EDTA.). The majority of the E-selectin microspheres exhibited a rolling interaction thus revealing force-sensitive interactions between the probing molecule (E-selectin) and the tissue sections (FIG. 3). For this Figure, E-selectin microspheres were perfused over colon papillary carcinoma tissue in the DBTA assay. Images for a single particle were captured every 2 seconds and overlaid to create the composite image. The arrow heads show transit of a rolling microsphere at different time points, and the scale bar indicates 10 μm. Few interactions were observed when the assay was done in the presence of EDTA, a divalent cation chelator, which is known to diminish the activity of E-selectin.

Dynamic biochemical tissue analysis allows tight control over the force exerted on the bond between the probe molecule and the tissue section; altering the force alters the result of the assay.

The DBTA assay allows the investigator to easily alter the force that is exerted on the bond between the probe molecule and the tissue section. The force on the bond is directly related to the size of the particle and the shear stress. Thus we illustrate the ease of altering the force by simply altering the size of the particle used in the assay and the shear stress. For this study, E-selectin microspheres (polystyrene particles prepared with 10 μg/ml recombinant mouse E-Selectin-IgG chimera for 2 hrs and blocked with 1% BSA) of 10 μm and 15 μm size were perfused over tissue sections derived from invasive colon adenocarcinoma at various shear stresses. Data are mean±SEM for n=3. Means that do not share a letter are significantly different (One way ANOVA coupled with Tukey's multiple comparison test, P<0.0001). As shown in FIGS. 4 and 5A-5D, the number of interacting particles was clearly a function of both particle size and shear stress dramatically illustrating that the results of a biochemical tissue analysis are coupled to the force exerted on the bond between the probe molecule and the tissue section. Images were acquired for adhesion of 15 μm (FIGS. 5A and 5B) and 10 μm (FIGS. 5C and 5D) E-selectin microspheres perfused over tissue sections derived from invasive colon adenocarcinoma at 0.5 (right panels) and 1.5 dynes/cm² (left panels). All images were acquired over the same tissue area. Each white dot on the images is an adhering microsphere, and the scale bars indicate 100 μm. Traditional biochemical tissue analysis does not allow systematic exploration of the relationship between the force on the bond and the results of the assay. The DBTA assay not only illustrates the importance of this relationship, but also provides a simple method to explore this association. Thus, by providing a means to characterize this relationship, a novel pathway for the development of biotechnologies (e.g. novel diagnostics and prognostics) is established.

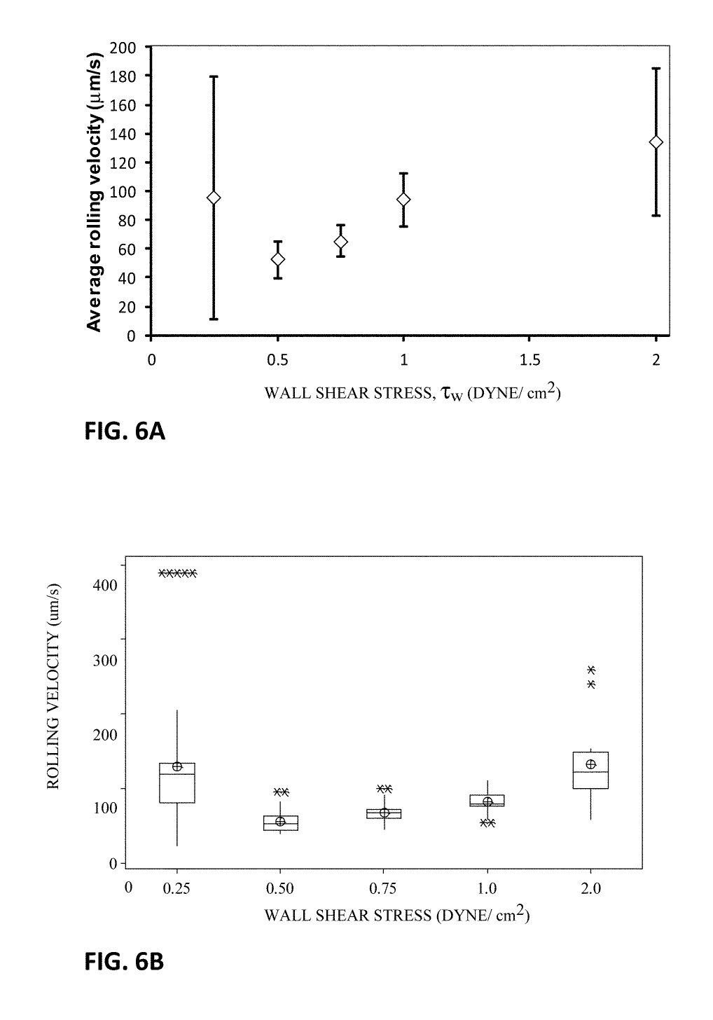

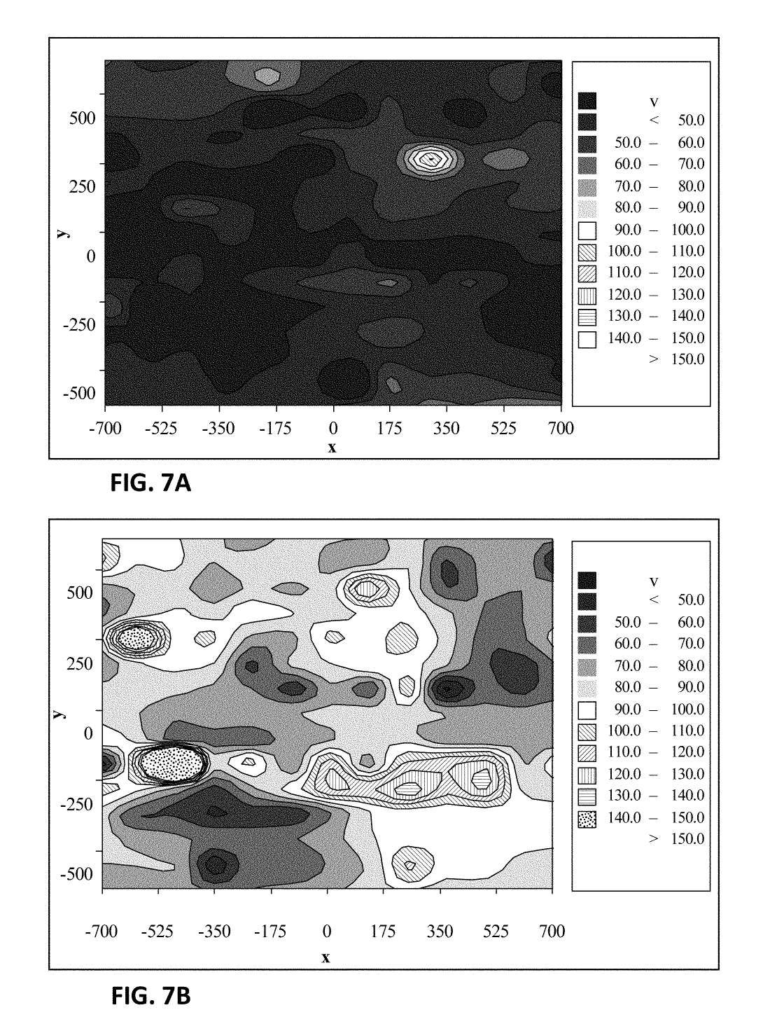

Here we demonstrate that DBTA is successfully employed for another probe molecule, L-selectin, and that altering the force by simply altering the shear stress again allows systematic exploration of the relationship between the force on the bond and the results of the assay. The DBTA assay was performed with 10 μm diameter particles for a range of shear stresses. As shown in FIGS. 6A-7B the rolling velocities of the microspheres interacting with a colon cancer tissue are clearly a function of wall shear stress, again dramatically illustrating that the results of a biochemical tissue analysis are coupled to the force exerted on the bond between the probe molecule and the antigen(s) in the tissue section. For this study, L-selectin microspheres (protein-A coated polystyrene particles prepared with 30 μg/ml recombinant human L-Selectin-IgG chimera for 1 hr and blocked with 1% BSA) of 10 μm diameter were perfused at various shear stresses over a tissue section derived from a human signet ring cell colon carcinoma sample. Rolling velocities shown in FIG. 6A are presented as mean+/−root-mean-square error (RMSE). FIG. 6B shows the same data illustrated in FIG. 6A instead as a whisker and box plot. Whiskers (solid vertical lines) indicate ranges of the lowest and highest values. Grey shaded boxes represent the interquartile range (the two mid-quartiles, i.e., the quartiles covering the 25%-75% data range). Target symbols indicate the mean, lines segmenting the interquartile range are the medians, and starred values are outliers. All data are from n=32 microspheres on a single tissue section except for 2.0 dyne/cm², in which n=16 microspheres (attributed to fewer rolling events compared to other shear stresses). Note the stabilization of rolling velocities at 0.5 dyn/cm² illustrates the hallmark catch-slip bond behavior of selectins (probe) and their ligands (antigen). Similar to data in FIGS. 2 and 9, hIgG microspheres failed to attach to the tissue section at any shear stress.

FIGS. 7A and 7B utilize the same data from FIGS. 6A and 6B, which were exported from image analysis of 32 microspheres at each shear stress, using Tracker (to generate x position, y position, and rolling velocity data) into Excel, which was then copied to Minitab 16 for contour plot generation. Note that the dramatic difference in the contour plots (FIG. 7A vs 7B), with a relatively moderate change in the shear stress, again clearly demonstrates that the force exerted on the probe antigen bond(s) significantly influences the results of a biochemical tissue analysis.

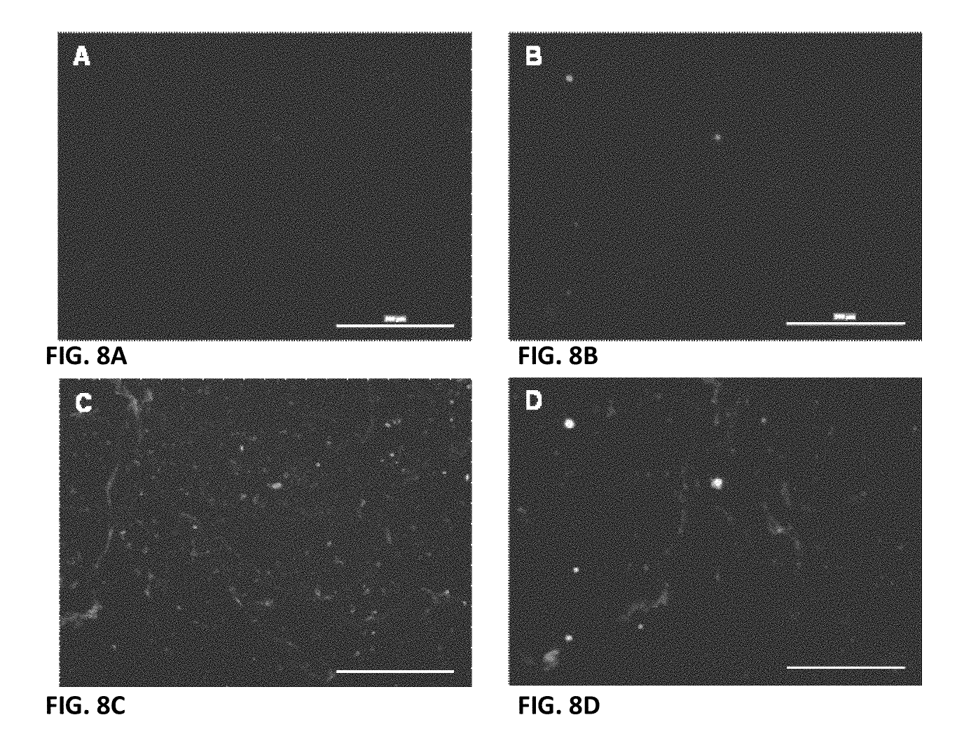

Thus, the same data analysis of biophysics of adhesive interactions can be reported in different manners to reveal nuances in the data relative to statistical parameters (FIGS. 6A and 6B) or to the spatial organization of the tissue itself (FIGS. 7A and 7B). By comparison, traditional biochemical tissue analysis does not allow systematic exploration of the relationship between the force on the bond and the results of the assay (FIG. 8A-8D). For this study, serial tissue sections of the sample used in FIGS. 6A-7B were immunolabeled in traditional biochemical assays with human L-selectin-hIgG Fc chimera (i.e., the probe used to functionalize microspheres in FIGS. 6A-7B) or hIgG isotype control, followed by secondary anti-hIgG AlexaFluor 568 antibody. FIGS. 8A and 8B show that the original images of L-selectin-stained and hIgG-stained serial sections do not show specific signals. As seen in FIG. 8C, upon image enhancement of FIG. 8A to amplify the signal-to-noise ratio, greater positive (red) staining with L-selectin can be observed compared to FIG. 8D the image-enhanced hIgG isotype control of the original image FIG. 8B, which was amplified under the same conditions as FIG. 8C. Although it may be possible to demonstrate statistical differences between FIG. 8C and FIG. 8D, the differences are subtle when compared to the results obtained from a dynamic biochemical tissue analysis with L-selectin conjugated to microspheres. In addition, these signals obtained with the traditional immunohistochemistry assay do not demonstrate the complexity of the dynamic signal responses under fluid flow conditions whereby forces are applied to the L-selectin/L-selectin-ligand bonds (i.e., probe/antigen bond). Scale bar=500 μm in all images.

To provide evidence that DBTA can be used as a potential diagnostic/prognostic assay, E-selectin-IgG microspheres were perfused over tissue sections derived from colon carcinoma. For these data, E-selectin or h-IgG microspheres (15 μm polystyrene particles prepared with 10 μg/ml recombinant mouse E-Selectin-human Fc chimera or hIgG for 2 hrs and blocked with 1% BSA) were perfused over tissue sections from signet ring carcinoma (Sc); mucinous adenocarcinoma with necrosis (Mc); or normal colon tissue (Nc), at 1 dynes/cm² in the DBTA assay. Data are mean±SEM for n=3. Means that do not share a letter are significantly different with P<0.001 (One way ANOVA with Tukey's multiple comparison test). As shown in FIG. 9, microspheres coated with E-selectin exhibited significantly higher levels of interaction, relative to negative control hIgG microspheres, with tissue sections derived from signet ring colon carcinoma (Sc) and mucinous adenocarcinoma (Mc). Microspheres coated with E-selectin exhibited dramatically higher interactions with colon carcinoma derived tissue sections (Sc and Mc) compared to normal tissue (Nc). This reveals that, at least for two cancer types, DBTA discriminates between cancerous and non-cancerous tissue. This is quite remarkable given that the DBTA assay used here has yet to be optimized. Interestingly, higher level of E-selectin microsphere interactions were observed over the more aggressive Sc derived tissue than with the less aggressive Mc derived tissue demonstrating that DBTA appears to distinguish between cancer types.

Combined these results reveal that a DBTA can reveal statistical differences in Sc and Mc tissue compared to normal tissue and distinct differences Sc and Mc tissue sections thus providing proof of concept/reduction to practice of a DBTA based diagnostic/prognostic assay.

Note that the tissues used in DBTA remain unstained, so they can be utilized post-test in other assays with stains or fluorescent labels.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A method of analyzing a tissue from a subject comprising:
contacting a tissue with a probe conjugate comprising a probe molecule conjugated to an inert surface;
applying a known force to at least one of the probe conjugate or the tissue, wherein the force tends to cause or enhance the interaction between the probe molecule and tissue, tends to disrupt the interaction between the probe molecule and the tissue, or tends to both cause or enhance the interaction and disrupt the interaction between the probe molecule and the tissue; and
quantifying at least one of the resulting interaction or disruption of interaction between the probe conjugate and the tissue.

2. The method of claim 1 wherein the force includes an associative force and a disruptive force, wherein the associative force and the disruptive force are the same force or distinct forces.

3. The method of claim 1 wherein the force is not sufficient to disrupt an interaction between the molecular probe and the tissue.

4. The method of claim 1 wherein the force is not sufficient to cause an interaction between the molecular probe and the tissue.

5. The method of claim 1 wherein the probe molecule is chosen from the group consisting of an amino acid, a peptide, a polypeptide, an antibody, fragments of antibodies, a protein, a glycoprotein, a carbohydrate, a polysaccharide, a nucleotide, a polynucleotide, oligonucleotide, a RNA, a DNA, a peptide nucleic acid, a lipid, a glycoplipid, a siRNA, a miRNA, a proteoglycan, a small molecule and combinations thereof.

6. The method of claim 1 wherein the inert surface is in the form selected from the group consisting of a particle, a generally planar surface, an end portion of a projection extending from a base structure, and combinations thereof.

7. The method of claim 6 wherein the particle has a widest diameter of about 100 μm.

8. The method claim 6 wherein the particle is generally spherical.

9. The method of claim 6 wherein the projection has a length along the axis extending from the base structure and a width wherein the length is in a range from about 500 nm to about 10 μm and the width is in a range from about 1 μm to about 100 μm and the tip portion ranges in diameter from 1 nanometer 100 micrometers.

10. The method of claim 6 wherein the probe molecule is conjugated to the generally planar surface by micropatterning.

11. The method of claim 1 wherein the composition of the inert surface is chosen from the group consisting of a polystyrene, a micelle, a proteoglycan, a biodegradable polymer, a ceramic, a protein, a synthetic polymer, a biological polymer, a magnetic material, an electrically active material, a liposome, a polymersome, a quantum dot, a metal containing polymer, a metal, an ultrasound bubble, an optically active particle, and combinations thereof.

12. The method of claim 1 wherein the composition of the inert surface is a fluorescent polystyrene particle.

13. The method of claim 1 wherein the force is a force field including the naturally occurring gravitational force, a mechanical force, a centrifugal force, an electrical force, a magnetic force, an optical force, an acoustic force or a combination thereof.

14. The method of claim 13 wherein the force field is generated by at least one of a fluid flow, a magnetic field, an electric field, an optical force, acoustic force, or utilizes the force of gravity and combinations thereof.

15. The method of claim 1 wherein more than one force is applied to at least one of the probe conjugate or the tissue.

16. The method of claim 15 wherein the force is generated by a device that includes multiple channels.

17. The method of claim 1 wherein the force is generated by a microfluidic device.

18. The method claim 1 wherein quantifying includes at least one of enumerating the number of particles on the surface or characterizing the adhesive nature of the particles on the surface.

19. The method of claim 18 wherein at least one of the enumerating the number of particles on the surface or characterizing their adhesive nature are quantified using one of manual or automatic image analysis.

20. A method of diagnosing a disorder in a subject or predicting the outcome for a subject with a disorder, the method comprising:
acquiring a tissue sample from the subject and analyzing the tissue with the method of claim 1.

21.-22. (canceled)

23. A novel therapeutic design method for a subject comprising:
acquiring a tissue sample from the subject;
analyzing the tissue with the method of claim 1; and
providing a treatment to the subject based on the analysis of the tissue.

24. A method of guiding the therapeutic treatment of a subject comprising:
administering a treatment to a subject;
acquiring a tissue sample from the subject; and
evaluating the effectiveness of the treatment with the method as in claim 1.

25. The method of claim 24 further comprising:
adjusting the treatment in view of the evaluation of the treatment's effectiveness.

26. An analytical composition comprising a probe conjugate that includes a probe molecule conjugated to an inert surface.

27. The composition of claim 26 wherein the inert surface is chosen from the group consisting of polystyrene, a biodegradable polymer, a protein, a synthetic polymer, a biological polymer, a magnetic material, an electrically active material, a liposome, a polymersome, a quantum dot, a metal containing polymer, a metal, an ultrasound bubble, an optically active particle, and combinations thereof.

28. The composition of claim 26 wherein the inert surface is in the form selected from the group consisting of a particle, a generally planar surface, an end portion of a projection extending from a base structure, and combinations thereof.

29. The composition of claim 22 wherein the probe molecule is chosen from the group consisting of an amino acid, a peptide, a proteoglycan, a polypeptide, an antibody, fragments of antibodies, a protein, a glycoprotein, a carbohydrate, a polysaccharide, a nucleotide, a polynucleotide, a RNA, a DNA, a lipid, a glycoplipid, a siRNA, a miRNA, a small molecule and combinations thereof.
